(19) United States
(12) Patent Application Publication
(10) Pub. No.: US 2019/0247288 A1
(43) Pub. Date: Aug. 15, 2019

(54) Nasal strip adhesive remover

(71) Applicant: Jorge Ramirez Arellano, Rossville, GA (US)

(72) Inventor: Jorge Ramirez Arellano, Rossville, GA (US)

(21) Appl. No.: 15/932,158

(22) Filed: Feb. 15, 2018

(51) Int. Cl. A61K 8/22; A61Q 19/10

(52) U.S. Cl. CPC A61K 8/22; A61Q 19/10

(57) ABSTRACT

This invention pertains to the use of the ingredients for the purpose of removing nasal strip adhesive from the nose after the nasal strip has been removed.

Above mentioned invention consists of the repurposing of an existing product (Carbamide peroxide 6.5%) and/or (Hydrogen peroxide 3%).

The Carbamide peroxide and/or the Hydrogen peroxide releases, upon application onto the nose, oxygen and helps soften, loosen and remove the sticky adhesive to allow the consumer to more easily wipe it from the nose.

### FIELD OF INVENTION

This invention pertains to the use of the ingredients for the purpose of removing nasal strip adhesive from the nose after the nasal strip has been removed.

### BACKGROUND

The advent of the nasal strip has led users to try all sorts of products to remove the adhesive left behind after the removal of the nasal strip.

So far most all of the products used by consumers are either abrasive or just will not remove the sticky adhesive.

Therefore, a need exists in the market for a product that aids in the removal of the adhesive left behind after removal of the nasal strip.

### BRIEF SUMMARY OF THE INVENTION

Above mentioned invention consists of the repurposing of an existing product (Carbamide peroxide 6.5%) and or (Hydrogen peroxide stabilized 3%). The (Carbamide peroxide) and or (Hydrogen peroxide) releases, upon application onto the nose, oxygen and helps soften, loosen and remove the sticky adhesive to allow the consumer to more easily wipe it from the nose.

### BRIEF DESCRIPTIONS OF THE DRAWINGS

N/A

### DETAILED DESCRIPTION OF THE INVENTION

The name used herein for the active ingredient is for the purpose of describing the product but not intended to limit the ingredient to this term as it is also known as H2O2, dihydrogen dioxide, hydrogen dioxide, hydrogen oxide, oxydol, peroxide, carbamide peroxide, urea peroxide, hydrogen peroxide carbamide, urea hydrogen peroxide, urea compd. with hydrogen peroxide (1:1) hydrogen peroxide.

1. (canceled)

2. A method of removing nasal strip adhesive, comprising:
a) applying an effective amount of Carbamide peroxide and/or Hydrogen peroxide to the surface of the nose to loosen the adhesive left behind after removal of the nasal strip;
b) then wiping the surface of the nose from base to ridge thus removing the sticky/loose adhesive along with said peroxide.

3. The method of claim 2 wherein the Carbamide peroxide is 6.5%.

4. The method of claim 3 wherein the Hydrogen peroxide is a stabilized 3% solution.
